(19) United States
(12) Patent Application Publication
(10) Pub. No.: US 2014/0173769 A1
(43) Pub. Date: Jun. 19, 2014

(54) System for Monitoring Growth Conditions of Plants

(71) Applicants: Frederik Leyns, Oosterzele (BE); Cedrick Vandaele, Gent (BE); Fabio Fiorani, Juelich (DE); Pierre Lejeune, Dolembreux (BE)

(72) Inventors: Frederik Leyns, Oosterzele (BE); Cedrick Vandaele, Gent (BE); Fabio Fiorani, Juelich (DE); Pierre Lejeune, Dolembreux (BE)

(73) Assignee: BASF PLANT SCIENCE COMPANY GMBH, Ludwigshafen (DE)

(21) Appl. No.: 13/981,130

(22) PCT Filed: Jan. 17, 2012
(86) PCT No.: PCT/IB12/50222

§ 371 Date: Feb. 5, 2014

Related U.S. Application Data
(60) Provisional application No. 61/435,381, filed on Jan. 24, 2011.

(30) Foreign Application Priority Data
Jan. 24, 2011 (EP) 11151858.5
Jan. 24, 2011 (EP) 11151863.5

(51) Int. Cl. G01N 27/22; A01G 27/00; A01H 1/04

(52) U.S. Cl. CPC G01N 27/223; A01H 1/04; A01G 27/00
USPC 800/260; 800/298; 324/658; 47/79

(57) ABSTRACT

A system (110) for monitoring growth conditions of a plurality of plant containers (112) is disclosed. The system (110) has a transport system (118) for transporting the plant containers (112). Each plant container (112) comprises at least one growing medium (114) and preferably at least one plant specimen (116). The system (110) further comprises at least one measurement position (130) having at least one contactless capacitive humidity sensor (132). The system (110) is adapted to successively transport the plant containers (112) to and from the measurement position (130). The system (110) is further adapted to measure the humidity of the growing medium (114) of the plant containers (112) in the measurement position (130) by using the contactless capacitive humidity sensor (132).

### FIELD OF THE INVENTION

The invention relates to a system and a method for monitoring growth conditions for a plurality of plant containers. The invention further relates to a tracking method for tracking growth conditions of a plurality of plant specimens. The invention further relates to a method for breeding plants, a method for improved growing of plants for phenotyping, for selecting the most desired genotypes based on phenotype scoring, and to a method for rapid analysis of stress resistance of growing plants. The invention further relates to a use of a contactless capacitive humidity sensor in a process for breeding plants, a use of a contactless capacitive humidity sensor in a drought screen and a use of a contactless capacitive humidity sensor for measuring water content in plant containers. The invention further relates to a method for providing a population of plant specimens and a population of plant specimens produced by the method.

Systems, methods and uses of this kind may be applied in all fields of agricultural research and manufacturing and in all fields of chemical and/or biological technology related to plants or plant specimens. Preferably, the systems and methods according to the present invention may be applied to the technical field of testing of plants and testing of methods for treatment of plants, such as one or more of: testing and/or evaluation of optimum growth conditions; testing of resistance of plants against specific types of stress; testing of specific fertilizers and/or nutrients; the selection and/or breeding of plants having one or more desired properties; the testing of the effect and/or effectiveness of specific treatments, such as treatments of the plants or plant specimens with fertilizers and/or pesticides. However, other applications of the present invention are possible.

### RELATED ART

Traditionally, in the technical field of farming and plant breeding for various purposes, the determination and/or control of optimum growth conditions has been one of the most important skills of a successful farmer or breeder. However, even the most talented and diligent farming in many cases could not prevent the plants from being subject to varying or uncontrollable conditions, such as climatic variations, changing properties of the growing medium or other uncontrollable external influences. These variations in external influences, however, in many instances are detrimental with regard to the possibility of comparing specific breeding results, such as comparing the effect of a certain treatment of plants and/or comparing different types of plants.

Due to these reasons, a number of techniques have been developed over the recent years, which allow for a more precise determination and/or control of the growth conditions of various types of plants. Thus, WO 2004/068934 A2 discloses a process for breeding plants, comprising growing plants of a species in an array of containers charged with growing medium of uniform characteristics in an environment of controlled climatic conditions with controlled supply of nutrients and feed water. The process further comprises a changing of the positions of the containers within the environment as required to ensure at least substantially uniform exposure of all plants in the containers to conditions in the environment. The process further comprises the step of selecting plants for further breeding for commercial use by comparing the phenotypic characteristics of the plants.

Similarly, EP 1 433 377 A1 discloses an apparatus suitable for use in conjunction with a container in which one or more plants are growing and having associated with it a device for receiving an enquiry signal and automatically responding by transmitting a unique identifier signal. The apparatus comprises a transporter means by which a container may be supported for moving the container, a means for transmitting the enquiry signal, a means for recording the identifier signal as a digital output and a computer means to which the digital output is supplied for storage of the data in predescribed format in a database for manipulation to afford comparison of data related to a container.

The named prior art documents mainly relate to systems suited for providing and/or controlling growth conditions to a large number of plants. However, even under controlled environmental conditions, the growth conditions may vary from plant to plant and from container to container, since, e.g., the need of water or liquid or nutrients may be dependent on the specific plant. Thus, as an example, the humidity in each growth container may vary despite of identical environmental conditions. Therefore, a large number of analytical techniques have been developed, which are suited for determining the actual growth conditions of the plants.

Besides optical techniques, other types of sensors for detecting growing conditions are known. Thus, conventional weighing techniques are known. Such weighing techniques for monitoring the humidity of the soil, however, require complex technical systems, in order to put the containers on a weighing machine. Subsequently, the container has to come to an equilibrium, in order to be weighed. Thus, these systems slow down the process of high throughput screening. Furthermore, the measured weight needs to be converted to a water content. Therefore, the dry content of the soil and the weight of the container need to be determined, and all handling of the container thereafter must be performed in a way that no soil is lost.

Further, CN 0201349436 Y discloses an automatic flower watering controller, consisting of a flower pot, a humidity sensor, a controller and a water pipe, wherein the humidity sensor is embedded in the soil of the flower pot. When moisture content in the soil is reduced, the humidity sensor sends out water lacking signals to the controller, in order to realize flower watering.

Besides humidity sensors being embedded in the soil, other types of humidity sensors are known. E.g., WO 2010/031773 A1 discloses a plant growth substrate water content measuring device to determine a value of water content in a substrate for growing plant material. The device comprises a first electrode and a second electrode and a control means connected to the first electrode and the second electrode, the control means comprising detecting means for registering a capacitance between the first electrode and the second electrode and a calculating means to deduce from the registered capacitance a value of the water content in the substrate.

Similarly, WO 93/13430 A1 discloses a system for non-invasive monitoring of the hydration state of a plant, the system comprising a timing capacitor comprising a plurality of conductive elements adapted for mounting on a plant part to sense the hydration state capacitance of the plant part. Further, a capacitance-to-frequency convertor is electrically connected to the timing capacitor and provides the timing capacitor with an electrical potential.

Further, in C.M.K. Gardner et al: Soil Water Content Measurement with a High-Frequency Capacitance Sensor, Journal of Agricultural Engineering Research (1998) 71, 395-403, details of capacitive monitoring of soil water content are disclosed. Specifically, the basic principles of capacitive humidity measurement using probes and/or electrodes which are inserted into the soil are disclosed, including calibration techniques.

Meanwhile, capacitive sensors for moisture measurements are commercially available for a wide variety of applications. E.g., in J. Mergl: Process Automation and Optimization with Online Moisture Measurement, available from Feuchtemesssysteme and lndustriekomponenten, Germany or online via www.acoweb.de, commercially available sensor systems are disclosed which may be used for online moisture measurements in various types of bulk solids, such as for quality control or monitoring process flows.

In DE 19710591 A1, the use of a transmitter and a receiver are disclosed. By inductive coupling through a plant container containing soil, the water content of the container is measured.

In U.S. Pat. No. 3,626,286, a meter for measuring moisture in soil is disclosed. The meter uses two probes spaced apart with soil between the probes. The probes may be insulated plates of metal or a flat insulated cable made of a plurality of conductors. The circuit has an ultrasonic oscillator which transmits a signal to the probes, which function as a variable capacitor depending upon moisture content of the soil. Further, the use of the moisture sensor for growing plants is disclosed.

In EP 0 392 639 A2, a method for measuring the moisture of water content of a substrate or growing product for growing plants of which at least a part consists of artificial material is disclosed. A capacitance value of said substrate is measured between two or more electrodes.

In WO 2004/109238 A1, multi-functional sensors are disclosed, comprising metal layers arranged as resistors around a central pair of resistors separated by a humidity sensitive polymer. Further, a plant management system using the sensors is disclosed, in order to monitor moisture in the soil adjacent to the plant. The sensor is clipped onto a sensor stake and pushed into the ground.

In EP 1 564 542 A1, a plant growth analyzing system and method are disclosed. An image acquisition system for acquiring images and a conveying mechanism for conveying a plurality of plants are used. Further, the recording of environmental conditions such as temperature and humidity is disclosed.

In US 2010/0286973 A1, a method for targeting trade phenotyping of plant breading experiment is disclosed. In the method, soil data for at least one location are collected and applied to a crop model performing environmental monitoring of the at least one location to generate environmental data.

In WO 2010/031780 A1, an improved plant breeding system is disclosed. The document specifically relates to a method for automated high throughput analysis of plant phenotype and plant genotype in a breeding system.

In L. Cattivelli et al.: “Drought tolerance improvement in crop plants: An integrated view from breeding to genomics”, FIELD CROPS RESEARCH, vol. 105, no. 1-2 2008, pages 1-14, general observations of drought effects in breeding plants are disclosed.

However, despite the progress that has been made in the field of monitoring and control of the growth conditions, the devices and methods known in the art still exhibit some major shortcomings.

Humidity sensors using contact probes, such as the device disclosed by CN 0201349436 Y, are disadvantageous in that only a limited space within the soil of the plants is monitored in view of humidity. Furthermore, this type of contact probes may cause a damage of the roots of the plants, and the repeated probing, comprising a putting of the probe in and out of the soil, will loosen the soil. Further, every time the probe is taken out of the soil, some soil will come out with the probe, which might, after repeated measurements, leave the container half empty or might lead to a cross-contamination of the containers.

Contactless methods and systems, such as many capacitive measurements as the measurement systems disclosed in WO 2010/031773 A1, typically are designed for monitoring and/or controlling the water content in one substrate material. It is even pointed out that continuous measurements of one and the same substrate are advantageous, in order to avoid disruptions. These findings, however, lead to the fact that, in known systems, a large number of humidity sensors have to be provided, in order to monitor and control each and every plant specimen. Thus, high-throughput screening of a large number of plants and/or growing conditions, in view of conventional techniques, is extremely expensive and complex.

### Problem to be Solved

It is therefore an object of the present invention to provide systems and methods which at least partially avoid the disadvantages and shortcomings of the systems and methods known from the prior art. Specifically, the systems and methods should enable a more precise breeding, monitoring, conditioning and testing of a large number of plants and/or growth conditions, at a significantly reduced technical and financial effort.

### SUMMARY OF THE PRESENT INVENTION

This problem is solved by the systems, methods and uses as claimed in the independent claims. Preferred embodiments of the invention, which may be realized in an isolated way or in arbitrary combination, are disclosed in the dependent claims.

In a first aspect of the present invention, a system for monitoring growth conditions of a plurality of plant containers is disclosed. The system may be a single apparatus or may comprise a number of two or more apparatuses, which may be arranged in a centralized or de-centralized way. In case the system comprises more than one apparatus, the apparatuses may at least partially be interconnected by mechanical and/or electronical means or may at least partially function in an isolated way.

As used in the present specification, the term compromising or grammatical variations thereof are to be taken to specify the presence of stated features, integers, steps or components or groups thereof, but do not preclude the presence or addition of one or more other features, integers, steps, components or groups thereof. The same applies to the term having or grammatical variations thereof, which is used as a synonym to the term comprising.

As used in the present invention, the expression monitoring may refer to a detection and/or recording of one or more parameters, such as physical and/or chemical parameters. The parameters may be detected and/or recorded in an arbitrary way, such as by measuring one or more digital and/or analogue signals and/or by recording one or more pieces of information on a data storage device and/or a database and/or by providing a hard copy of the parameters. Other types of detection and/or monitoring are possible.

As used in the present invention, the expression growth conditions may relate to any effect or influence, such as external influence that might have an impact on the growth of a plant. Thus, growth conditions may comprise one or more of the following conditions: a humidity of a growing medium, such as soil and/or hydroponics; the presence and/or concentration of one or more analytes and/or chemical compounds in the growing medium and/or the ambient air; a humidity content of the ambient air; a temperature of the growing medium; a temperature of the ambient air; amount of light; amount of space.

The monitoring of the growth conditions may imply a simple recording of one or more of the growth conditions and/or may even comprise a controlling and/or modification of the growth conditions. Thus, the term monitoring may imply an adjustment and/or regulation of one or more of the growth conditions.

The term plant container, as used in the present invention, may imply any type of container which is suited to at least partially hold a growing medium and/or a plant or plant specimen, such as by providing a mechanical support and/or a casing, which fully or partially surrounds the growing medium and/or the plant or plant specimen. The plant containers may be of arbitrary shape and may be selected from the group containing pots, bowls, cups, pods, or any other shape. Basically, the plant containers may at least partially surround the growing medium or may even be part of the growing medium itself. Thus, the growing medium at least partially may be solidified, in order to provide a mechanical protection and in order to prevent from disintegrating. Thus, the plant container may comprise an outer layer of the growing medium, which is solidified, whereas a further part of the growing medium is at least partially comprised in this outer layer. Other types of plant containers are possible.

The system or at least part of the system may be placed in a controlled environment, such as a greenhouse or any other environment in which at least one climate parameter may be controlled or even regulated at least to a certain degree, such as a temperature and/or a humidity of ambient air. The controlled environment, such as the greenhouse, may even be part of the system itself. The transport system and/or the measurement position may be located inside the controlled environment.

The system is adapted for monitoring the growth conditions of a plurality of plant containers. The plant containers may be part of the system. A plurality of at least two plant containers may be provided, preferably a plurality of at least five, most preferably at least ten or even at least one hundred plant containers may be provided or may be part of the system. Each plant container may comprise a specific amount of growing medium and at least one plant or plant specimen.

The system has a transport system for transporting the plant containers. The transport system may comprise any known means for transporting the plant containers, such as a system selected from one or more of the following: a conveyor, preferably a belt-conveyor or band-conveyor; a roller belt; a roller conveyor; a linear actuator, such as a motion state; a transport cart; a gripper; a crane; a robot. However, combinations of the named systems and/or other systems are possible. Preferably, the transport system is adapted for automatically transporting plant containers, preferably without the need of any human input or interaction. However, other types of transport systems are possible.

As outlined above, each container comprises at least one growing medium and preferably at least one plant specimen. As used herein, the term plant specimen may refer to any plant or part of a plant, such as roots, trunks, leaves, seeds, seedlings. Preferably, each plant container contains precisely one plant or plant specimen. However, other embodiments are possible. In the following, unless explicitly mentioned otherwise, the terms plant and plant specimen are used as synonyms, notwithstanding the fact that both terms may refer to one or more whole plants or parts thereof, such as roots, trunks, leaves, seeds, seedlings. Independent from the use of the singular or plural form, the terms plant, plants, plant specimen or plant specimens each may refer to one single plant or plant specimen on a plurality of plants or plant specimens.

The system further comprises at least one measurement position having at least one contactless capacitive humidity sensor. This measurement position may comprise a measurement station, which may or may not be part of the transport system or may be connected to the transport system, in order to allow for a successive transport of the containers to and from the measurement position. More than one measurement position may be provided. As used herein, the term “measurement position” denotes a position and/or apparatus of the system, in which or by which at least one measurement may be performed. However, other types of functionality may be comprised in the measurement position, such as control means and/or watering means, such as a watering station, recording means, computer means or other types of functionality or combinations thereof.

The measurement position, e.g. one or more apparatuses comprised by the system in the measurement position, has at least one contactless capacitive humidity sensor. As used herein, the term contactless refers to a means which not necessarily has to be in direct contact with the plant or plant specimen comprised in the containers. The contactless capacitive humidity sensor not necessarily needs to contact the growing medium nor the plant or plant part for the humidity measurement. Preferably, the system is designed such that, during an overall measuring cycle, no part of the contactless capacitive humidity sensor gets in contact with any part of the plant or plant specimen. Further, preferably, no part of the contactless capacitive humidity sensor gets in contact with the growing medium either. Preferably, the contactless capacitive humidity sensor is located outside the plant containers, without the need of sticking any part of the contactless capacitive humidity sensor into the growing medium at any time.

As used herein, the term capacitive humidity sensor refers to a sensor or sensor system being based on a capacitive measurement principle. Thus, as an example, the capacitive sensors disclosed in the above-mentioned publications by J. Mergl may be used. Preferably, the capacitive humidity sensor may be adapted to create an electric field, preferably an alternating electric field which at least partially percolates or permeates the growing medium, preferably the whole growing medium comprised in the container in the measurement position. From changes in the capacitance, induced by the humidity of the growing medium and optionally the plant or plant specimen, the sensor or the system may deduce the humidity of the growing medium and optionally the plant or plant specimen. This humidity might be provided in absolute values of a given physical unit, such as in g/cm³, or may be provided in any other way, such as by providing one or more parameters which are directly or indirectly correlated to the humidity such that the humidity may be derived directly or indirectly from these parameters.

The system is adapted to successively transport the containers to and from the measurement position. Specifically, the transport system may be adapted to provide this successive transport. A successive transport may imply that one or more than one plant container are transported to the measurement position to be measured by the humidity sensor, followed by at least one further plant container or group of plant containers, which are transported to the measurement position at a later point in time. Preferably, the system is adapted to transport the containers to the measurement position and from the measurement position in equal time intervals, such that a time interval between the transport of a first plant container to the measurement position and the transport of the successive plant container to the measurement position is equal for all plant containers. Other embodiments are possible. A single measurement position or a plurality of measurement positions may be provided. The transport may be performed in a stepwise fashion or in a continuous fashion or in a combination thereof.

The system further is adapted to measure the humidity of the growing medium of the containers in the measurement position by using the contactless capacitive humidity sensor. For this purpose, the capacitive humidity measurement methods, as outlined above, or as outlined in one or more of the named prior art documents, may be used. The results of the measurement of the humidity may be subject to a further processing by the system, such as a processing selected from: a displaying of the measurement result, a storing and/or recording of the measurement result, a storing of the measurement result in a database, an output of a hard copy of the measurement result. Combinations of the named possibilities and/or other possibilities are feasible.

As opposed to many of the prior art systems, an advantage of the present invention resides in the fact that a contactless capacitive humidity measurement is feasible. The system is adapted to determine the water content in the plant containers in a contactless way. E.g., the contactless capacitive humidity sensor may be adapted to create a dome-shaped measurement area, such that the water content of the volume within this dome-shaped area above, beneath or next to the contactless capacitive humidity sensor may be measured. The dome-shaped area of measurement may completely cover the area of the at least one plant container in the measurement position, such that the water content of the whole growing medium in the container may be measured, as opposed to known measurements using humidity probes. Further, complex calculations and/or measurements may be avoided, such as the calculation of water content from weight measurements. Further, by using the contactless measurement, the loss of soil or any other growing medium may be avoided. Further, disturbances of the soil structure or the structure of the growing medium are avoided, as well as potential damages to roots.

The system may be adapted to perform high throughput screening measurements, preferably in an automated way. The measurements may be performed fluently, without the need of complex measurement procedures, such as a limiting of reflections in optical systems.

As outlined above, the transport system may be designed in various ways. Preferably, the transport system may be or may comprise a closed loop system being adapted for repeatedly transporting all containers into the measurement position. As used herein, the expression closed loop system refers to a transport system being capable of transporting a plurality of plant containers in a predetermined order, the transport system being capable of repeatedly and successively transporting the plant containers into the measurement position in the predetermined order. Thus, preferably, the transport system comprises a transport circle of arbitrary shape, the transport circle being capable of repeatedly transporting each plant container to the measurement position by using a first section of the transport circle and transporting the plant container from the measurement position by using a second section of the transport circle, the second section being connected to the first section, preferably outside the measurement position. However, other transport systems are possible, such as transport systems using one or more robots or other transport apparatuses for transporting the plant containers into the measurement position.

Preferably, the system for monitoring growth conditions of the plurality of plant containers is adapted to transport each container into the measurement position at a predetermined point in time and/or in predetermined time intervals, preferably at least once a week or even once every day. This embodiment might be achieved e.g. by monitoring the position of each plant container and by adapting a transport velocity in such a way that the above-mentioned condition is fulfilled. Alternatively or additionally, the transport system may comprise a plurality of predetermined transport locations, each of which might be occupied by at least one plant container, such as predetermined floor spaces of a transport belt. The transport locations successively may be transported to the measurement position at predetermined time intervals, such as by tacting a new transport location into the measurement position as soon as a predetermined time interval has elapsed, such as a time interval of several seconds, minutes or even hours. The transport locations might contain specific platforms or floor spaces of the transport system, such as equally spaced platforms, wherein each plant container might be positioned on a platform. Other transport locations or other types of transport systems are possible.

In a preferred embodiment, the contactless capacitive humidity sensor is performing or may be adapted to perform the humidity measurement from a lower side of the plant containers through a bottom section of the plant containers. Thus, the contactless capacitive humidity sensor may be adapted to generate an electric field, such as an alternating electric field, which percolates the bottom section of the plant containers. E.g., as mentioned above, the contactless capacitive humidity sensor may be adapted to generate a dome-shaped electric field percolating the plant containers through the bottom section and, preferably, covering the whole content of the plant containers.

Preferably, the contactless capacitive humidity sensor may comprise one compact sensor unit, which may be located below the plant containers in the measurement position. Thus, a sensor unit as disclosed in the above-mentioned publications by J. Mergl may be used. However, the contactless capacitive humidity sensor may be or may comprise other types of sensors.

Preferably, the contactless capacitive humidity sensor is adapted to measure the humidity of the whole content of the plant containers, which means the whole content of at least the growing medium comprised in the respective plant container located in the measurement position. Additionally, the contactless capacitive humidity sensor may be adapted to measure the humidity of the plant being contained in the plant containers.

As mentioned above, the contactless capacitive humidity sensor preferably may be adapted to generate an electric field, preferably an alternating electric field. Preferably, the contactless capacitive humidity sensor may operate at 10 MHz to 300 MHz, preferably at 80 MHz to 150 MHz. These frequencies are well-suited to percolate typical materials of plant containers, such as plastic materials, clay, ceramic materials, stone, fabric or other materials which are typically used for plant containers. Further, these frequencies are well-suited for percolating typical growing media, such as soil, fabric, hydroponics or other growing media.

The contactless capacitive humidity sensor may be adapted to generate at least one measurement signal characterizing the humidity. This at least one measurement signal may be a single signal or a sequence of signals. The measurement signal may comprise an analogue and/or digital signal. The measurement signal may be an electrical signal, such as a voltage and/or current signal and/or a digital electrical signal. Preferably, the contactless capacitive humidity sensor may be adapted to generate at least one voltage signal, preferably a voltage signal from 0 VDC to 10 VDC and/or a current signal, preferably a current signal from 0 mA to 20 mA. However, other embodiments are possible.

As mentioned above, the transport system may be designed in various ways and may comprise one or more types of transport apparatuses. Preferably, the transport system comprises at least one transport belt. In this embodiment, preferably, the contactless capacitive humidity sensor may be mounted underneath the transport belt, preferably in the measurement position. However, alternatively or additionally, other types of transport apparatuses are feasible, as outlined above.

In addition to the measurement position, the system may further have at least one watering station, and the system may be adapted to add liquid to the growing medium in each plant container, preferably automatically. One or more watering stations may be provided. The watering station may at least partially be integrated into the measurement position or, alternatively or additionally, the system may comprise at least one separate watering station, independent from the measurement position.

As used herein, the term watering station refers to an apparatus of the system being adapted to add liquid to the growing medium. Thus, the watering station may comprise one or more liquid supplies and one or more orifices or other types of apparatuses being adapted to provide the liquid to the growing medium, such as a cube, a valve, a nozzle, a tap, a sprayer or any combination of the named apparatuses and/or other apparatuses.

Further, as used herein, the term liquid may refer to any substance at least partially being in the liquid state. Preferably, the term liquid refers to aqueous substances, such as pure water or water containing one or more ingredients, such as one of: salt, nutrients, fertilizers, pesticides. Thus, even saline water may be used and may be added to the plant containers. The adding of liquid to the growing medium in each plant container, preferably in each plant container when positioned in the watering station, may be performed automatically, semi-automatically or non-automatically, wherein an automatic adding of liquid is preferred, i.e. an adding of liquid without the necessity of human interference and/or intervention.

The system may be adapted to automatically control the humidity in each plant container or in the growing medium of each plant container. As used herein, the term control refers to an adjustment of the humidity to a predetermined level, preferably automatically. The system may even be adapted to regulate the humidity of the growing medium in each plant container. As used herein, the term regulate refers to a process in which an actual value of the humidity is compared with at least one predetermined target value, and, from the comparison, at least one actuating variable is generated, which has an impact on the humidity in the growing medium, such as an actuating variable acting on the watering station. However, other types of watering stations are feasible.

Preferably, the system may be adapted to add liquid to the growing medium in each plant container to a predetermined humidity level, preferably automatically. As mentioned above, this adding of liquid preferably may be performed in a controlled or even regulated way.

Preferably, the at least one predetermined humidity level may be adjusted, such as by a computer system and/or manually. Preferably, the predetermined humidity level may be adaptable individually for each plant container.

In a further preferred embodiment, the system may be adapted to automatically recognize a malfunctioning of the system by evaluating the humidity in at least one plant container, preferably in all plant containers. Preferably, the system may be adapted to automatically recognize a malfunctioning of the at least one optional watering station. Thus, the system may be adapted to recognize that the humidity level in one or more or preferably all of the plant containers is equal to or below a predetermined lower limit, and, thus, may be adapted to automatically recognize a malfunctioning of the watering station and/or the transport system transporting the plant containers to the watering station.

In case a malfunctioning is recognized, the system may further be adapted to take one or more predetermined safety measures, preferably automatically. Thus, the system may be adapted to perform one or more of the following actions in case a malfunctioning of the system, preferably a malfunctioning of the watering station, is recognized: output a warning, such as by displaying a warning and/or outputting at least one acoustic and/or visual warning signal and/or by notifying at least one further component of the system or at least one external component; stop the overall action of the system; stop the overall action of the transport system; record the malfunctioning, such as by recording the malfunctioning in a database, preferably by recording an entry comprising at least the point in time of the malfunctioning and/or the type of malfunctioning. However, alternatively or additionally, other types of safety measures may be taken, such as adjusting the amount of liquid added to each plant container, e.g. by temporarily increasing the amount of liquid added to the plant containers.

In a further preferred embodiment, the plant containers each may have at least one identifier. Preferably, these identifiers may be or may comprise one or more of the following identifiers: a barcode; a contactless electronic identifier, preferably at least one rapid frequency identification tag (RFID tag). However, alternatively or additionally, other types of identifiers are possible. Preferably, the at least one identifier comprises at least one contactless identifier, i.e. an identifier comprising at least one piece of information, which may be read from the identifier without any physical contact between a reading mechanism and the identifier. Each plant container may comprise one or more identifiers. The at least one identifier may be comprised in the plant containers, such as by integrating the identifier into a material of the plant containers and/or on a surface of the plant containers, preferably an outer surface, and/or by integrating the identifiers in an interior space of the plant containers, such as by implementing the identifiers into the growing medium inside the plant containers and/or by implementing the identifiers onto or into the plants contained in the plant containers. Alternatively or additionally, other types of implementation of the identifiers into the plant containers are possible. In general, the at least one identifier not necessarily has to be in physical contact with the plant container, but should be assigned to a respective plant container in any unambiguous way.

The system preferably is adapted to identify the identifiers. Thus, the system may comprise one or more identification apparatuses, such as one or more reading apparatuses, which may be located in one or more positions of the system, preferably in one or more locations of the transport system. Thus, the system may comprise at least one reading apparatus for reading the at least one identifier in the measurement position and/or in or close to the watering station. As used herein, the term reading refers to the detection of at least one piece of information contained in the at least one identifier, optionally comprising one or more steps of decoding the information.

Preferably, the system is adapted to identify the plant container presently being located in the measurement position. Alternatively or additionally, the system may be adapted to identify the respective plant container presently being located in the watering station and/or any other predetermined position of the system. This may be achieved by positioning at least one reading apparatus adapted for reading the electronic identifier of the respective plant container in the watering station and/or the measurement position. However, alternatively or additionally, other types of embodiments are possible.

Thus, the system may comprise at least one reading station separated from the measurement position and/or separated from the watering station, and, preferably, may be adapted to track the movements of the plant containers from this reading station, such as in a stepwise or continuous movement, in order to recognize the specific plant container presently being located in the measurement position and/or the watering station. By combining a transport information, which may be provided by the transport system or other parts of the system, with the information provided by the at least one reading station, a precise tracking of the plant containers and an information on a current position of each plant container may be retrieved.

In this embodiment or other embodiments, the at least one optional reading apparatus being adapted to read the at least one identifier may comprise one or more types of reading apparatuses. Thus, one or more optical reading apparatuses may be comprised, such as optical apparatuses for reading one or more barcodes assigned to the containers. Thus, one or more barcode readers may be comprised. Additionally or alternatively, other types of reading apparatuses may be present, such as RFID-readers or other types of contactless electronic identifier readers.

In a preferred embodiment, the at least one identifier may comprise at least one data storage device. Thus, at least one volatile and/or at least one non-volatile data storage device may be present in the identifier. The optional at least one reading station may be adapted to read information from the data storage device and/or to write information into the data storage device. Thus, it may be possible to write data back to the identifier. Thus, the at least one identifier may comprise a data storage, such as a storage chip, for data such as humidity data and/or plant identification. The data storage may be implemented into any kind of identifier, such as into a contactless identifier, e.g. an RFID chip, an electronic data carrier or an optical data carrier.

In a further preferred embodiment, the system may further have at least one monitoring system. The at least one monitoring system may be adapted to monitor the humidity of the growing medium in the plant containers, preferably in each plant container, preferably as a function of plant specimen and/or as a function of time.

Preferably, the system and more preferably the at least one monitoring system may comprise at least one recording apparatus, the recording apparatus being adapted to record the humidity of the growing medium in the plant containers, preferably in each plant container. Thus, a time development of the humidity of the growing medium in the plant containers may be recorded. Alternatively or additionally, the type of plant specimen may be recorded, and the humidity of the growing medium of the respective plant container comprising the respective plant specimen may be recorded.

The recording apparatus may comprise one or more data storage systems, such as one or more volatile and/or non-volatile data storage systems. Alternatively or additionally, the monitoring system may comprise one or more data processing systems, such as one or more computers, preferably one or more microcontrollers.

The at least one data processing system may comprise at least one database, the database being adapted to monitor the humidity of the growing medium in the plant containers, preferably as a function of plant specimen and/or as a function of time. Other types of monitoring systems are feasible. In this or in other embodiments of the present invention, the plant containers comprised in the system not necessarily have to be identical. Thus, different types of plant containers and/or different types of growing media and/or different types of plant specimens may be used. The monitoring system may have at least one database for recording various types of information, such as a database for recording the humidity of the growing medium in each plant container as a function of plant specimen and/or as a function of time.

Preferably, the system according to the invention may have at least one imaging system for capturing images of the plant specimens. Thus, the system according to the invention may have one or more imaging stations, which may be designed as separate imaging stations and/or as imaging stations which are at least partially integrated into the measurement position and/or the optional watering station and/or any other station. Thus, the imaging system may comprise one or more imaging sensors, such as optically sensitive CCD chips and/or CMOS chips and/or any other imaging chip. Additionally, the imaging systems each may comprise one or more imaging optical systems, such as one or more lenses, diaphragms, reflective elements such as mirrors and/or combinations of the named and/or other optical elements. Further, the imaging system may comprise one or more filter systems. The at least one imaging system may be adapted for one or more spectral wavelengths, such as wavelengths in the infrared or near-infrared spectral range and/or the visible range and/or the ultraviolet range. Additionally or alternatively to imaging systems being adapted for electromagnetic waves, imaging systems using other types of rays may be used, such as X-ray systems and/or particle imaging systems.

A capturing of the images may be performed in various ways. Thus, the capturing of the images may be performed in a purely electronic way, such as by storing imaging information electronically, e.g. by using one or more databases and/or one or more volatile or non-volatile data storage devices. Additionally or alternatively, the images may be displayed, such as by using a display unit. Again, alternatively or additionally, the images may be transferred to other devices and/or a printout of the images may be generated.

Further, the at least one imaging system or another part of the system according to the present invention may be adapted to perform an image analysis. Thus, one or more image processing units may be comprised in the system, preferably at least partially in the imaging system, which may be adapted for full or partial processing of the captured images. Thus, specific results may be derived from the captured images, such as color parameters and/or parameters characterizing a volume of the plants and/or other types of parameters, preferably automatically.

Preferably, the system according to the present invention may further have at least one measurement device for measuring at least one growth parameter of the plant specimens. Again, this at least one measurement device may at least partially be integrated into other devices of the system, such as into the measurement position and/or into the watering station and/or into the at least one imaging system. Additionally or alternatively, the system may comprise the at least one measurement device as separate device and/or as a stand-alone device, being separate from other apparatuses of the system according to the present invention. The at least one measuring system may use one or more physical and/or chemical measurement principles, in order to measure the at least one growth parameter of the plant specimens. Thus, optical principles may be used, such as by using the at least one imaging system disclosed above.

As already explained, from the captured images of the plant specimens, one or more growth parameters may be derived, such as one or more color parameters and/or a volume of the plant specimens and/or a root volume of the plant specimens and/or a plant height and/or a biomass of the plant specimens and/or a combination of the named and/or other parameters.

The system may further be adapted to record the growth parameter for each plant container in a database. Preferably, the at least one growth parameter is recorded in the database, which may comprise any type of suitable storage device, as a function of time and/or as a function of a plant specimen. As outlined above, the at least one growth parameter may comprise one or more parameters characterizing the growth of the plant specimen. The at least one growth parameter may preferably be chosen from: a height of the plant specimen; a width of the plant specimen; a color parameter or color parameters of the plant specimen; a number of leaves; at least one structure of the plant specimen; a presence of flowers in the plant specimen; a parameter characterizing the volume of the biomass of the plant specimen; a parameter characterizing the biochemical content of the plant specimen and/or the growing medium inside the plant container; a parameter characterizing the root growth in the plant specimen. However, other types of parameters and/or combinations of the named parameters and/or other parameters are possible.

In a further aspect of the present invention, a method for monitoring growth conditions of a plurality of plant containers is disclosed. Each plant container comprises at least one growing medium and preferably at least one plant specimen. The plant containers are successively transported to and from at least one measurement position, such as by using a transport system, preferably a transport system as disclosed above. The humidity of the growing medium of the plant containers in the measurement position is measured by using at least one contactless capacitive humidity sensor.

With regard to potential embodiments of the method according to the present invention, reference may be made to the above-mentioned system for monitoring growth conditions of a plurality of plant containers. Thus, the method according to the present invention may be performed by using a system according to the present invention. Thus, reference may be made to the embodiments and definitions disclosed above. However, other types of systems may be used.

In a preferred embodiment, the method according to the present invention is performed such that a water consumption of each plant specimen is monitored and preferably recorded. Thus, the water consumption of each plant specimen may be derived from successive measurement of the humidity, such as humidity measurements in one measurement cycle and a humidity measurement in at least one subsequent measurement cycle, in which the plant container is positioned again in the measurement position. Preferably, this water consumption may be derived from these measurements, in consideration of the liquid added to the plant container in the optional at least one watering station, such as by calculating a net consumption of water or liquid for each plant specimen. The recording, again, may be performed by using at least one volatile or non-volatile data storage device and/or by using at least one database. The calculations may be performed by using at least one data processing apparatus, such as by using at least one computer. Thus, the system according to the present invention and/or the method according to the present invention may use one centralized computer and/or a de-centralized computer system having more than one computer. The data processing apparatus may comprise one or more software packages, in order to perform one or more steps of the present method, such as a calculation of the water consumption.

In a further aspect of the present invention, a tracking method for tracking growth conditions of a plurality of plant specimens is disclosed. The plurality of plant specimens are growing in a growing medium, which is at least partially located inside a plurality of plant containers. The tracking method uses the method for monitoring growth conditions, as disclosed above or as disclosed in one or more of the embodiments disclosed below, for controlling the humidity in each plant container. Within the tracking method, the humidity in each plant container is stored in a database, preferably as a function of time and/or as a function of plant specimen. Thus, as used herein, the term tracking method for tracking growth conditions refers to a method, which, in addition to simply monitoring the growth conditions, makes use of at least one database, in order to generate a tracking record of the humidity in each plant container, such as for later comparison of the growing results with the tracking record of the growing conditions.

Further, in addition to the at least one humidity measurement for each plant container, the database may contain further information. Thus, as outlined above, the humidity in each plant container might be stored as a function of time and/or as a function of plant specimen. Additionally or alternatively, the at least one database may comprise further data. Thus, at least one growth parameter for each plant specimen may be recorded in the database, preferably as a function of time and/or as a function of plant specimen. With regard to potential growth parameters, reference may be made to the disclosure of potential growth parameters as listed above.

Besides simply recording data, the tracking method may further comprise one or more steps of evaluating the data or part of the data comprised in the at least one database. Thus, the tracking method may further comprise at least one method step in which, by comparing the growth parameters and the soil humidity of the plant containers, an optimum soil humidity is derived.

Further, additionally or alternatively to one or more evaluation steps, the tracking method may further comprise one or more testing steps, in which the reaction of the plant specimens to specific growing conditions is tested. Thus, the tracking method may comprise one or more steps in which a drought test and/or a water use efficiency test is performed. In this at least one drought test and/or at least one water use efficiency test, a variety of plant specimens is subjected to a lack or reduced amount of water over a period of time, wherein the plant specimens' reaction to the lack of water or reduced amount of water is recorded. Thus, again, one or more growth parameters and/or the time development of this at least one growth parameter may be recorded and/or evaluated, in order to qualify and/or quantify the plant specimens' reaction to the lack of water or reduced amount of water.

As an example, a greenness parameter may be used and may be recorded over a period of time, during which the drought test and/or water use efficiency test is performed, and the greenness index and/or the time development of the greenness index may be used to qualify and/or quantify the plant specimens' reaction to the drought test and/or water use efficiency test. Within this drought test and/or water use efficiency test, the variety of plant specimens may comprise a variety of different plant specimens, which are subjected to the same drought test and/or water use efficiency test, or, alternatively or additionally, a variety of plant specimens of the same type may be subjected to different types of drought tests and/or water use efficiency tests, such as by subjecting the variety of plant specimens of the same type to a lack or reduced amount of water to a different degree, in order to evaluate the sensitivity of the plant specimens' reaction to the lack or reduced amount of water. Other types of drought tests and/or water use efficiency tests are possible and known to the skilled person.

The drought resistance and/or water use efficiency of the plant specimens may be evaluated and/or monitored. Thus, such as by evaluating specific growth parameters, e.g. the greenness index, the resistance of the plant specimens to a lack of water or reduced amount of water may be compared and/or evaluated qualitatively and/or quantitatively. By comparing the added amount of liquid with the plants' drought resistance, the water use efficiency of the plant specimens may be monitored.

In a further aspect of the present invention, a method for breeding plants is disclosed. As used herein, the term breeding refers to any type of reproduction of plants, including the selection of plants or plant specimens with specific desired characteristics for propagation. Further, the term plant breeding may comprise more complex techniques, such as the selection of at least one specific phenotypic and/or genotypic characteristics, such as by evaluating specific plant parameters and/or growth parameters and/or genetic characteristics. In addition to the selection of specific plants or plant parts, the breeding may comprise one or more other steps, such as the steps of generating seedlings of selected plants.

The method for breeding plants according to the present invention comprises growing a plurality of plants of at least one species in a plurality of plant containers charged with a growing medium of uniform characteristics in an environment of controlled climatic conditions, with controlled supply of liquid. The plurality of plant containers may comprise an array of plant containers or a row of plant containers, charged with the growing medium.

As used herein, the term uniform characteristics refers to growing media in different plant containers, which are identical as far as possible with common techniques, such as growing media which are taken from the same supply of a growing medium. Thus, at least macroscopically and, more preferably, chemically, the growing conditions provided by the growing media in different plant containers are identical at least to the point of experimental uncertainty.

Further, as used herein, the term environment of controlled climatic conditions refers to an environment of the plant containers in which at least one climatic parameter is adjusted to one or more specific, pre-determined values. Thus, the environment of controlled climatic conditions may comprise an environment, in which the ambient temperature is adjusted to at least one predetermined temperature, which might be static or might be subjected to a time development. The control may comprise a control to a specific temperature value within an experimental uncertainty of less than 1° K or less, such as to 0.5° K. The controlled climatic conditions may comprise a regulation of the climatic conditions, such as by using at least one controller or regulator, in order to regulate the climatic conditions to at least one pre-determined value.

Further, as used herein, the term controlled supply of liquid refers to the fact that the supply of liquid to each plant container is performed in a pre-determined way, such as by using the system according to the present invention in one or more of the embodiments disclosed above. Thus, the controlled supply of liquid may comprise a pre-determined rate of liquid supply to each plant container. Thus, as outlined above, one or more watering stations may be used in order to control the supply of liquid.

Further, the method for breeding plants according to the present invention comprises a changing of the positions of the plant containers within the environment as required to ensure at least substantially uniform exposure of all plants in the plant containers to conditions in the environment. In other words, in case there are N potential positions of the plant containers in the environment, the method is performed in such a way that the amount of time spent in position i, with i=1 to N, is substantially equal for all plant containers, which, preferably, means that the variation in between the containers is less than 1 h, preferably less than 10 min and more preferably less than 1 min. However, the amount of time each plant container is positioned in the potential positions may vary in between different positions.

Again, this changing of positions may be performed by using a system according to the present invention and as disclosed in one or more of the embodiments above. Preferably, at least one transport system is used. By using this method, variations of the growing conditions of the plants in the plant containers which are due to different locations in the environment may be reduced to a minimum.

The method for breeding plants according to the present invention further comprises the step of selecting plants for further breeding or for commercial use by comparing the phenotypic characteristics of the plants. As used herein, the term phenotypic characteristics refers to at least one observable characteristics or trait of the plant or plant specimen, such as at least one morphological parameter or the time development of the at least one morphological parameter. Thus, the at least one phenotypic characteristics which may be used for comparison of the plants may comprise one or more of the growth parameters and/or one or more of the morphological parameters and/or the time development of these parameters, such as one or more of the growth parameters and/or one or more of the morphological parameters and/or one or more of the resistances, such as the resistance to at least one drought test.

Within the method for breeding plants, the containers are successively transported to and from a measurement position by at least one transport system, such as by using the system as disclosed above. The humidity of the growing medium of the plant containers is measured in the measurement position by using at least one contactless capacitive humidity sensor, preferably the contactless capacitive humidity sensor according to one or more of the embodiments of the contactless capacitive humidity sensor, as disclosed above within the context of the system according to the present invention.

In a further aspect of the present invention, a method for improved growing of plants for phenotyping, for selecting the desired genotypes based on phenotype scoring, is disclosed. As used herein, the term phenotyping refers to the monitoring of one or more phenotypic characteristics of plants or plant specimens. Further, as used herein, the term genotype refers to the genetic constitution of the plants or plant specimens or at least one part thereof. The term phenotype scoring refers to a qualitative or quantitative comparison of the results of the phenotyping as disclosed above, such as to a qualitative and/or quantitative comparison of one or more phenotypic characteristics. This scoring may be performed on a quantitative scale, such as by using at least two classes for classifying the phenotypic characteristics of the plants or plant specimens.

The method for improved growing of plants comprises at least one step of displacing the plants automatically during the growing cycle, so as to avoid extended exposure to a particular micro-environment. Thus, reference may be made to the method for breeding plants as disclosed above and to the at least one step of changing the positions of the plant containers of this method. Specifically, a system according to the present invention may be used, which comprises one or more transport systems. Thus, reference may be made to the embodiments disclosed above.

The method for improved growing of plants further comprises at least one step of measuring a humidity of a growing medium of the plants by using at least one contactless capacitive humidity sensor. With regard to the definitions and/or potential embodiments of the contactless capacitive humidity sensor, reference may be made to the system according to the present invention in one or more of the embodiments as disclosed above.

The method for improved growing of plants further comprises at least one step of controlling the humidity. As used herein and as defined above, the term controlling the humidity refers to the adjustment of the humidity to at least one predetermined level, which might be constant or time-dependent. The adjustment may comprise a simple adjustment to the at least one predetermined value or may even comprise a regulating of the humidity to the at least one predetermined value. For controlling the humidity, the at least one watering station as disclosed above may be used.

In a further aspect of the present invention, a method for rapid analysis of stress resistance of growing plants is disclosed.

As used herein, the term stress resistance of growing plants refers to the degree of capability of specific plants or plant specimens of continuing their growing process in a more or less unaffected way despite of detrimental growing conditions, such as lack of water, salty water, lack of nutrients, non-optimum ambient temperatures or combinations thereof. Thus, the term stress refers to non-optimum growing conditions, such as one or more of the non-optimum growing conditions mentioned before.

The term rapid analysis refers to a quantitative and/or qualitative evaluation of the stress resistance of at least one growing plant, preferably the comparison of stress resistances of different types of growing plants, on a short timescale, such as on a timescale comprising no more than 5 growing cycles, preferably no more than 2 or most preferably no more than 1 growing cycle or even less, such as a timescale of 5 months or less, preferably 3 months or less or even 1 month or less.

The method for rapid analysis of stress resistance of growing plants according to the present invention comprises at least one step of growing the plants under stress conditions. As outlined above, these stress conditions may comprise any type of non-optimum growing conditions or combinations thereof. Further, the method according to the present invention comprises at least one step of measuring a humidity of a growing medium of the plants by using at least one contactless capacitive humidity sensor.

Preferably, the at least one capacitive humidity sensor may be designed as disclosed above in the context of the system according to the present invention. Further, the method for rapid analysis of stress resistance of growing plants comprises at least one step of analyzing the stress resistance of the plants based on the humidity. Thus, the stress resistance may be evaluated qualitatively and/or quantitatively for one plant or a plurality of plants, by at least partially evaluating the humidity measured by the at least one contactless humidity sensor. Thus, the water consumption of the at least one plant may be evaluated, in order to qualify and/or quantify the stress resistance of the at least one plant. Alternatively or additionally, at least one other type of parameter may be used to qualify and/or quantify the stress resistance, and the humidity measured by the at least one contactless capacitive humidity sensor may be used to quantify and/or qualify the degree of stress exposure of the plants.

In a further aspect of the present invention, a use of a contactless capacitive humidity sensor in a process for breeding plants is disclosed. Again, with regard to the term breeding, reference may be made to the above-mentioned definition. The use may further comprise the use of the system for monitoring growth conditions of a plurality of plant containers according to one or more of the embodiments disclosed above.

In a further aspect of the present invention, a use of a contactless capacitive humidity sensor in a drought screen is disclosed. With regard to the term drought, reference may be made to the disclosure of one or more of the methods described above. Thus, a drought screen may comprise a testing of a plurality of plants under a plurality of different drought conditions. Again, the use may further comprise the use of the system for monitoring growth conditions of a plurality of plant containers according to one or more of the embodiments disclosed above.

In a further aspect of the present invention, a use of a contactless capacitive humidity sensor for measuring water content in plant containers is disclosed. Again, the use may further comprise the use of the system for monitoring growth conditions of a plurality of plant containers according to one or more of the embodiments disclosed above.

In a further aspect of the present invention, a method for providing a population of plant specimens is disclosed. The population preferably has a low plant-to-plant variability. This aspect is based on the finding that, for performing specific tests and/or comparisons, a uniform population of plant specimens is desirable. Thus, for evaluating the phenotypic effect of certain effectors, a population of plant specimens should be provided, which preferably exhibit a low plant-to-plant variability, such as a low plant-to-plant variability of at least one growth parameter. Thus, in other words, all plants of the population preferably should be more or less similar, in order to reduce the impact of plant-to-plant variations on the testing results.

Thus, in this further aspect of the present invention, a method for providing a population of plant specimens is disclosed. The population of plant specimens preferably has a low plant-to-plant variability. This method preferably uses the system according to one or more of the embodiments disclosed above, i.e. the system for monitoring growth conditions of a plurality of plant containers. Alternatively or additionally, the method preferably may use a contactless capacitive humidity sensor. However, other systems and/or sensors may be used additionally or alternatively, such as non-contactless humidity sensors.

The method comprises the following steps which preferably may be performed in the given order. However, other sequences are possible. Further, one or more of the method steps may be performed in a different order and/or may be performed in a time-parallel or timely overlapping fashion. Again, one or more of the steps may be performed repeatedly.

Firstly, the method comprises at least one step of determining standard watering conditions leading to a predetermined breeding result, preferably an optimum breeding result. These standard watering conditions may comprise watering of at least one growing medium of the plant specimens to at least one predetermined level, which may be constant or which may vary from at least one upper level down to at least one lower level, such as by using a sequence of watering and drying steps. As disclosed below, the predetermined breeding result may be a breeding result of the plant specimens having at least one growing parameter, such as a leaf area, a body mass or a combination of growing parameters. In this regard, reference may be made to the above-mentioned growing parameters. Preferably, the at least one predetermined breeding result is an optimum breeding result, such as an optimum or maximum leaf area or an optimum or maximum biomass of the plant specimens. However, other standard watering conditions are possible.

In a further method step, at least one drought condition including watering conditions below, the watering conditions of the standard watering conditions are determined. Thus, these drought conditions may comprise an average watering being below an average watering of the standard watering conditions as disclosed above. Alternatively or additionally, the drought conditions may comprise longer periods without re-watering of the growing medium. Again, alternatively or additionally, the drought conditions may comprise re-watering or watering up to at least one upper level below the at least one upper level of the standard watering conditions and/or a drying of the growing medium down to at least one lower level being below the lower level of the standard watering conditions.

A further step of the method comprises breeding of a population of plant specimens in at least one plant container comprising at least one growing medium, by using the drought conditions as determined above. This population may comprise at least two, preferably three, four or more plant specimens, preferably of the same species. These plant specimens may be kept in the same plant container, such as by breeding a plurality of plant specimens in one or more rows of plant specimens. Alternatively or additionally, a plurality of plant containers may be used, each plant container comprising at least one growing medium and at least one plant specimen.

Preferably, during breeding of the population of plant specimens, at least one contactless capacitive humidity sensor is used for monitoring the drought conditions. However, alternatively or additionally, other types of humidity sensors may be used.

Preferably, the breeding of plant specimens takes place by using the drought conditions before flowering of the plant specimens. Preferably, after flowering, the standard watering conditions are used.

Preferably, at least one growth parameter of the plant specimens is chosen as a measure of the impact of watering conditions on the breeding result. With regard to the potential growth parameters applicable in this embodiment, reference may be made to the above-mentioned growth parameters. Preferably, at least one leaf area of the plant specimens and/or at least one biomass of the plant specimens may be used. The standard conditions may be chosen such that an average of the growth parameters of the population assumes a maximum. Thus, the standard conditions may be derived from at least one pre-breeding experiment, such as an experiment subjecting a plurality of plant specimens to different watering conditions, determining a watering condition leading to a growth parameter assuming the maximum value. These watering conditions leading to the maximum value may be chosen as standard watering conditions.

Preferably, the drought conditions comprise a watering of the growing medium such that the growing medium is watered up to at least one predetermined upper level, preferably a maximum capacity of the at least one growing medium. A re-watering is performed as soon as a humidity of the growing medium has decreased to at least one predetermined lower level. Thus, one or more watering cycles may be used, comprising a watering step watering the growing medium to the at least one upper level, followed by at least one drying step, during which the growing medium dries down to the at least one predetermined lower level. The drought conditions may comprise one or more drought cycles. Preferably, the drought conditions comprise at least two drought cycles, wherein in each cycle watering up to the at least one predetermined upper level and a subsequent decrease down to the at least one predetermined lower level takes place.

The drought conditions generally may comprise any sub-standard watering conditions. Preferably, the drought conditions are chosen such that the drought is strong enough to slow or even stop growth of the plants. This effect, however, should be fully reversible and should not result in permanent injury or damage to the plants. Thus, the drought level preferably should be chosen strong, but not too strong. When too strong, drought may cause permanent injuries and even higher variability. The drought conditions preferably may comprise a watering of the growing medium to a time-averaged value of 20% to 80% as compared to the standard conditions. Preferably, the drought conditions comprise a watering of the growing medium to a time-averaged value of 40% to 70% as compared to the standard conditions. As used herein, the term “time-averaged value” refers to a measurement of the value over a period of time, such as over several days. Thus, periods of drought and periods of re-watering may be comprised by time-averaging over these periods to form one common value. Thus, the time-averaged values may be target values to be reached at the end of the overall treatment. Astonishingly, it was discovered that a population of plant specimens produced by the method according to one or more of the above-mentioned embodiments, being bred under drought conditions, typically exhibits a lower plant-to-plant variability as compared to populations being bred under standard conditions. This will be outlined in more detail in the embodiments disclosed below. Again, for breeding the plant specimens, a contactless capacitive humidity sensor and/or a system as disclosed above is highly advantageous, since the use of this type of sensors and/or system significantly facilitates a high-throughput screening.

Thus, in a further aspect of the present invention, a population of plant specimens produced by the method according to one or more of the embodiments disclosed above is proposed.

As discussed above, a population of this type preferably may be used for testing one or more effector conditions. Thus, in a further aspect of the present invention, a method for determining the phenotypic effect of at least one effector condition is disclosed. The method comprises subjecting the population of plant specimens produced by the method according to one or more of the embodiments disclosed above to the at least one effector condition. Further, the method comprises determining at least one growth parameter of the plant specimens.

As used herein, the term “effector condition” refers to any internal and/or external influence that might have an impact on one or more phenotypic characteristics of the plant specimens. Thus, as an example, the at least one effector condition might comprise at least one genetic effector condition, such as the amount of expression of one or more specific genes of the plant specimens. Thus, an overexpression or a down-regulation of one or more genes, preferably as compared to a wild-type plant specimen and/or to a standard type plant specimen, may be comprised. Alternatively or additionally, the at least one effector condition might comprise one or more external conditions, such as biotic or abiotic stress, preferably with the exception of water stress. Thus, a biotic stress might be subjecting the plant specimen to one or more biotic influences, such as an influence by microorganisms and/or vermin and/or other plants. An abiotic stress, which might be applied additionally or alternatively, might comprise any type of stress due to external growth conditions, such as subjecting the plant specimens to light having a specific wave length and/or a specific intensity, subjecting the plant specimens to specific temperatures, subjecting the plant specimens to specific physical growing conditions in general and/or any combination of the named conditions.

The method for determining the phenotypic effect of at least one effector condition may further comprise subjecting at least two plant specimens of the population to different effector conditions, i.e. two effector conditions being distinct from each other with regard to at least one effector condition, wherein the growth parameters of the at least two plant specimens are compared.

The methods and uses according to the various aspects of the present invention preferably may be performed or may be implemented by using at least one system according to the present invention, i.e. by using at least one system for monitoring growth conditions of a plurality of plant containers, as disclosed above and/or by using at least one contactless capacitive humidity sensor. Thus, with regard to optional aspects of the methods according to the present invention, reference may be made to the optional embodiments of the system as disclosed above and/or as will be disclosed in more detail in the description of potential embodiments disclosed below.

The system, the methods and the uses according to the present invention provide a large number of advantages over known devices and methods. Thus, the system and methods according to the present invention allow for a precise testing of the plants' reactions to specific environmental conditions in a very controlled way, by substantially excluding other, unintended influences, such as the influence of the positioning of the plant containers within the environment and, thus, by excluding the influence of the micro-environment of the plant. The system, methods and uses according to the present invention are e.g. very useful for testing transgenic plants for the effect of a specific gene which is over- or underexpressed or even knocked down. On the other hand, the system and methods can be used to evaluate stress resistances, such as a resistance against a drought stress and/or salt stress and/or any other type of stress.

Further, additionally or alternatively, water use efficiency or any other characteristics of the plants may be evaluated. Stress resistance measurements may be based on humidity measurements, such as by using the well-known fact that a plant or plant specimen, which uses less water and, thus, evaporates less water, typically is in a worse physical condition than a plant or plant specimen using more water.

One or more of the methods disclosed above may be based on the fact that, when there is salt in the water, the plant has difficulties to absorb water and, thus, the physical conditions of the plant typically deteriorate. Thus, by monitoring the physical condition of the plant and/or by monitoring the humidity and/or the water consumption, specific properties of the plant or plant specimen, such as the stress resistance, may be monitored. Further, one or more of the methods and/or systems disclosed above may be used in order to study the capability of a specific plant or plant specimen to keep absorbing water under high moisture content of the surrounding air. Thus, the system according to the present invention and/or the method according to one or more of the methods according to the different aspects of the present invention may be adapted to monitor the moisture content of the surrounding air as one or more additional parameters, preferably as a function of time.

Summarizing the above-mentioned ideas of the invention, the following items are proposed:
Item 1: A system for monitoring growth conditions of a plurality of plant containers, the system having a transport system for transporting the plant containers, each plant container comprising at least one growing medium and preferably at least one plant specimen, the system further comprising at least one measurement position having at least one contactless capacitive humidity sensor, the system being adapted to successively transport the plant containers to and from the measurement position, the system further being adapted to measure the humidity of the growing medium of the plant containers in the measurement position by using the contactless capacitive humidity sensor.
Item 2: The system according to the preceding item, wherein the transport system is a closed loop system being adapted for repeatedly transporting all containers into the measurement position.
Item 3: The system according to the preceding item, the system being adapted to transport each plant container into the measurement position at a predetermined point in time and/or in predetermined time intervals.
Item 4: The system according to one of the preceding items, wherein the contactless capacitive humidity sensor is performing the humidity measurement from a lower side of the plant containers through a bottom section of the plant containers.
Item 5: The system according to one of the preceding items, wherein the contactless capacitive humidity sensor is adapted to measure the humidity of the whole content of the plant containers.
Item 6: The system according to one of the preceding items, the transport system having a transport belt, wherein the contactless capacitive humidity sensor is mounted underneath the transport belt.
Item 7: The system according to one of the preceding items, the system further having at least one watering station, the system being adapted to add liquid to the growing medium in each plant container, preferably automatically.
Item 8: The system according to the preceding item, wherein the system is adapted to add liquid to the growing medium in each plant container to a predetermined humidity level, preferably to a predetermined humidity level being adaptable individually for each plant container.
Item 9: The system according to one of the preceding items, the system being adapted to automatically recognize a malfunctioning of the system by evaluating the humidity, preferably a malfunctioning of the watering station.
Item 10: The system according to one of the preceding items, the plant containers each having at least one identifier, preferably at least one barcode and/or at least one contactless electronic identifier, preferably at least one RFID tag, the system being adapted to identify the plant container presently being located in the measurement position.
Item 11: The system according to one of the preceding items, the system further having at least one monitoring system, the monitoring system being adapted to monitor the humidity of the growing medium in the plant containers, preferably as a function of plant specimen and/or as a function of time.
Item 12: The system according to the preceding item, the monitoring system having at least one database for recording the humidity of the growing medium in each plant container as a function of plant specimen and/or as a function of time.
Item 13: The system according to one of the preceding items, the system further having at least one imaging system for capturing images of the plant specimens.
Item 14: The system according to one of the preceding items, the system further having at least one measurement device for measuring at least one growth parameter of the plant specimens.
Item 15: The system according to the preceding item, the system further being adapted to record the growth parameter for each plant container in a database.
Item 16: The system according to one of the two preceding items, the at least one growth parameter being chosen from: a height of the plant specimen; a width of the plant specimen; a color parameter of the plant specimen; a number of leaves; at least one structure of the plant specimen; a presence of flowers in the plant specimen; a parameter characterizing the volume of the biomass of the plant specimen; a parameter characterizing the biochemical content of the plant specimen and/or the growing medium inside the plant container; a parameter characterizing the root growth of the plant specimen.
Item 17: A method for monitoring growth conditions of a plurality of plant containers, wherein each plant container comprises at least one growing medium and preferably at least one plant specimen, wherein the plant containers are successively transported to and from at least one measurement position, wherein the humidity of the growing medium of the containers in the measurement position is measured by using at least one contactless capacitive humidity sensor.
Item 18: The method according to the preceding item, wherein the system according to one of the preceding items referring to a system for controlling growth conditions is used.
Item 19: The method according to one of the preceding method items, wherein a water consumption of each plant specimen is monitored and preferably recorded.
Item 20: A tracking method for tracking growth conditions of a plurality of plant specimens, wherein the plurality of plant specimens are growing in growing medium inside a plurality of plant containers, wherein the method according to one of the preceding method items is used for controlling the humidity in each plant container, wherein the humidity in each plant container is stored in a database, preferably as a function of time and/or as a function of plant specimen.
Item 21: The tracking method according to the preceding item, wherein further at least one growth parameter for each plant specimen is recorded in the database, preferably as a function of time and/or as a function of plant specimen.
Item 22: The tracking method according to one of the preceding method items referring to a tracking method, wherein a drought test and/or a water use efficiency test is performed in which a variety of plant specimens is subjected to a lack or reduced amount of water over a period of time, wherein the plant specimens' reaction to the lack of water or reduced amount of water is recorded.
Item 23: The tracking method according to the preceding item, wherein the drought resistance and/or water use efficiency of the plant specimens is monitored.
Item 24: A method for breeding plants which comprises growing a plurality of plants of at least one species in a plurality of plant containers charged with growing medium of uniform characteristics in an environment of controlled climatic conditions, with controlled supply of liquid and changing the positions of the plant containers within the environment as required to ensure at least substantially uniform exposure of all plants in the plant containers to conditions in the environment, and which process further comprises the step of selecting plants for further breeding or for commercial use by comparing the phenotypic characteristics of the plants, wherein the plant containers are successively transported to and from a measurement position by a transport system, wherein the humidity of the growing medium of the plant containers in the measurement position is measured by using at least one contactless capacitive humidity sensor.
Item 25: A method for improved growing of plants for phenotyping, for selecting the most desired genotypes based on phenotype scoring, the method comprising: displacing the plants automatically during their growing cycle so as to avoid extended exposure to a particular micro-environment; measuring a humidity of a growing medium of the plants by using at least one contactless capacitive humidity sensor; and controlling the humidity.
Item 26: A method for rapid analysis of stress resistance of growing plants, the method comprising: growing the plants under stress conditions; measuring a humidity of a growing medium of the plants by using at least one contactless capacitive humidity sensor; and analyzing the stress resistance of the plants based on the humidity.
Item 27: Use of a contactless capacitive humidity sensor in a process for breeding plants.
Item 28: Use of a contactless capacitive humidity sensor in a drought screen.
Item 29: Use of a contactless capacitive humidity sensor for measuring water content in plant containers.
Item 30: A method for providing a population of plant specimens, the population of plant specimens preferably having a low plant-to-plant variability, the method preferably using the system according to one of the preceding items referring to a system for monitoring growth conditions of a plurality of plant containers, the method comprising: determining standard watering conditions leading to a predetermined breeding result, preferably an optimum breeding result; determining drought conditions including watering conditions below the standard watering conditions; breeding a population of plant specimens in at least one plant container comprising at least one growing medium, by using the drought conditions.
Item 31: The method according to the preceding item, wherein, during breeding of the population of plant specimens, a contactless capacitive humidity sensor is used for monitoring the drought conditions.
Item 32: The method according to one of the two preceding items, wherein the breeding of plant specimens takes place by using the drought conditions before flowering of the plant specimens, wherein afterwards preferably the standard watering conditions are used.
Item 33: The method according to one of the three preceding items, wherein at least one growth parameter of the plant specimens is chosen as a measure for the impact of watering conditions on the breeding result, wherein the standard conditions are chosen such that an average of the growth parameter of the population assumes a maximum.
Item 34: The method according to one of the four preceding items, wherein the drought conditions comprise a watering of the growing medium such that the growing medium is watered up to at least one predetermined upper level, wherein a re-watering is performed as soon as a humidity of the growing medium has decreased to at least one predetermined lower level.
Item 35: The method according to the preceding item, wherein the drought conditions comprise at least two drought cycles, wherein in each cycle a watering up to the at least one predetermined upper level and a subsequent decrease down to the at least one predetermined lower level takes place.
Item 36: The method according to one of the six preceding items, wherein the drought conditions comprise a watering of the growing medium to a time-averaged value of 20% to 80% as compared to the standard conditions, preferably to a time-averaged value of 40% to 70% as compared to the standard conditions.
Item 37: A population of plant specimens produced by the method according to one of the seven preceding items.
Item 38: A method for determining the phenotypic effect of at least one effector condition, the method comprising subjecting the population of plant specimens according to the preceding item to the at least one effector condition and determining at least one growth parameter of the plant specimens.
Item 39: The method according to the preceding item, wherein at least two plant specimens of the population are subjected to different effector conditions, wherein the growth parameters of the at least two plant specimens are compared.

### SHORT DESCRIPTION OF DRAWINGS

In the following, further potential details and features of the invention are disclosed in view of preferred embodiments, preferably in connection with the dependent claims. The features disclosed in the preferred embodiments may be realized in an isolated way or in any arbitrary combination. The invention is not restricted to the preferred embodiments. The embodiments are depicted in the figures in a schematic way. Identical reference numbers in the figures refer to identical, similar or functionally identical elements.

In the drawings:

FIG. 1 shows a top view of a system for monitoring growth conditions of a plurality of plant containers;

FIG. 2 shows a side view of a measurement position of the system according to FIG. 1; and

FIGS. 3 and 4 show comparisons of plant populations bred under normal conditions and under drought conditions.

### PREFERRED EMBODIMENTS

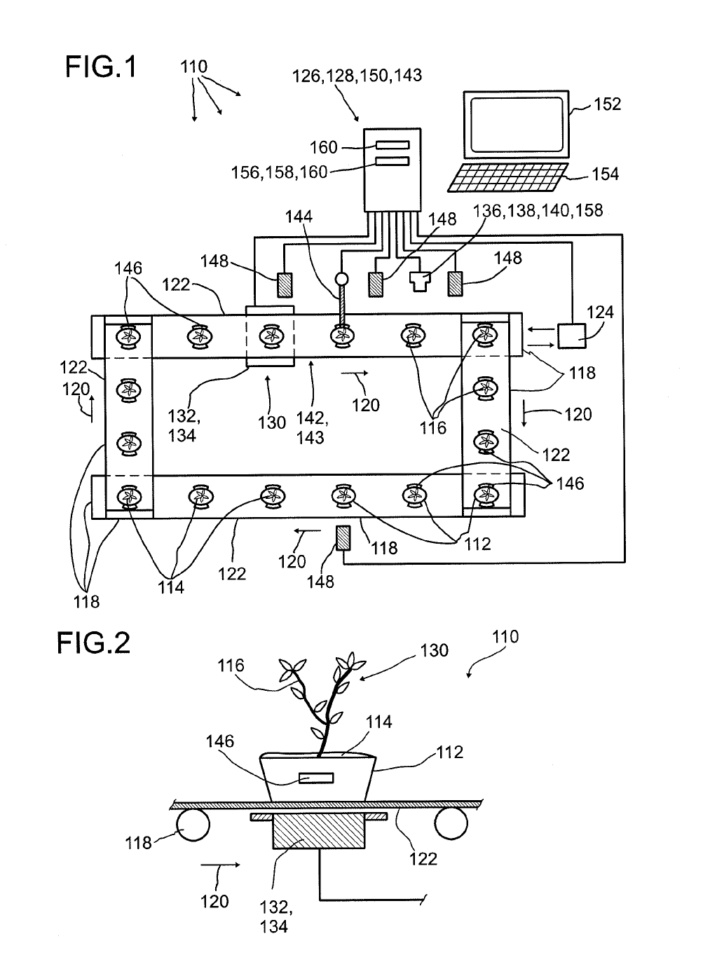

In FIG. 1, a top view of a system 110 for monitoring growth conditions of a plurality of plant containers 112 is depicted. Each plant container 112 comprises a growing medium 114 and at least one plant specimen 116.

The system 110 further comprises at least one transport system 118, which may be designed to transport the plant containers 112 in a transport direction 120. In the preferred embodiment depicted in FIGS. 1 and 2, the transport system 118 comprises transport belts 122. However, other types of transport systems 118 are feasible, additionally or alternatively. The transport system 118 in this preferred embodiment may be designed as a closed loop system, being capable of repeatedly transporting all plant containers 112 into one or more positions, such as in a transport in a clockwise sense in FIG. 1.

The transport system 118 may further comprise one or more transport controllers 124, as schematically depicted in FIG. 1. The at least one transport controller 124 may be connected or may be part of a centralized or decentralized system controller 126, such as a system controller 126 having one or more data processing devices 128. The transport controller 124 may be adapted to control the transport of the plant containers 112, such as by controlling the motion of one or more actuators and/or drive controllers, such as one or more belt drivers. Other embodiments are feasible.

The system 110 further comprises at least one measurement position 130. This measurement position 130, which may comprise one or more measurement stations, comprises at least one contactless capacitive humidity sensor 132. As depicted in FIG. 2, this contactless capacitive humidity sensor 132 may comprise a probe 134. Preferably, a probe 134 of the type “Feuchtemess-Sensor, type (D)MMS” by ACO Feuchtemesssysteme and Industriekomponenten, 79793 Wutoschingen-Horheim, Germany, may be used. The probe 134 may be installed under the transport belt 122.

The whole system 110 may be placed inside a greenhouse. The measurement position 130 may be adapted to assess the humidity, such as the pot water content, of all plant containers 112. The probe 134 may provide a permanent monitoring means to present a regular status of all plant specimens 116 present in the greenhouse.

The measurement position 130 may be followed by one or more further measurement devices 136, such as one or more optical imaging systems 138, e.g. one or more camera systems 140. In FIG. 1, the measurement device 136 schematically is positioned downstream of the probe 134. However, alternatively or additionally, other embodiments are feasible. E.g., the probe 134 may be positioned at an exit of the imaging system 134.

The system 110 may further comprise one or more watering stations 142, such as one or more watering stations 142 having one or more supply systems 144 for adding at least one liquid to the plant containers 112. The watering station 142 as depicted in FIG. 1 is schematically positioned after the measurement device 136. However, other positions are feasible, additional or alternatively.

The system 110 may further comprise at least one monitoring system for monitoring the humidity of the growing medium 114 in the plant containers 112, such as a function of plant specimen 116 and/or as a function of time. In the setup disclosed in FIG. 1 or other setups according to the present invention, this monitoring system may comprise the measurement position 130 and/or the contactless capacitive humidity sensor 132, as well as the system controller 126 and/or parts thereof. In FIG. 1, the monitoring system is denoted by referential 143. However, other types of monitoring systems 143 are feasible.

The system 110 may further comprise one or more identifiers 146, such as one or more identifiers 146 connected to each plant container 112 and/or to each plant specimen 116. Preferably, the identifiers 146 each comprise at least one contactless identifier, such as a barcode or, more preferably, at least one rapid frequency identification tag (RFID tag) and/or any other contactless electronic identifier.

The system 110 may further comprise at least one reader 148 adapted for reading information stored in the identifiers 146, such as an RFID reader and/or a barcode reader. In the schematic embodiment shown in FIG. 1, readers 148 are positioned in the measurement position 130 and/or the watering station 142 and/or comprised in the at least one measurement device 136 and/or positioned in any other way. Thus, the readers 148 may be adapted to identify the plant container 112 and/or the plant specimen 116 positioned in one or more of the measurement positions and/or the watering station 142 and/or in a position being monitored by the at least one measurement device 136 and/or in any other position of the system 110.

As depicted in FIG. 1, the components of the system 110, such as the probe 132, the watering station 142, the measurement device 136 or the reader 148, may be connected to the centralized or de-centralized system controller 126, such as to the data processing device 128. The system controller 126 may comprise one or more evaluation devices 150, which may be hardware and/or software implemented. The system controller 126 may be further adapted to comprise one or more data input and/or data output devices, such as one or more display devices and/or keyboards 154 and/or any other type of user interface. The data processing device 128 may further be connected to one or more further devices, such as to a computer network and/or the internet.

The system 110, preferably the system controller 126, may be adapted to check on the humidity status of all plant containers 112 and/or plant specimens 116 and/or growing media 114 at predetermined points in time, such as on a regular or irregular basis, preferably weekly. Thus, the system 110 may be adapted to weekly check on the water status and/or water use of all plants present in the greenhouse.

The system 110 may be adapted to estimate if the water regime is sufficient for the plants, such as by taking into account that the water consumption or, generalized, liquid consumption of all plants or plant specimen 116 may vary during the year. The system 110 may further be adapted to take appropriate action, such as an adjustment of the watering timing and/or the watering amounts, such as by controlling the humidity inside each plant container 112 to at least one predetermined level.

The system 110 may further be adapted to perform at least one failsafe routine. Thus, the system 110 may be adapted to detect mechanical problems in some parts of the transport system 118 and/or to detect a malfunctioning of the transport system 118 and/or the watering station 142. This way, an accidental under-watering of the plants may be avoided.

The system 110 and/or the system controller 126 may further comprise at least one database 156. The system controller 126 may be adapted to monitor the humidity of the growing medium 114 in each plant container 112.

The system controller 126 and/or the measurement device 136 may further comprise additional components for determining, preferably measuring, at least one growth parameter of the plant specimens 116. Thus, the imaging system 138 may comprise or may be connected to at least one image evaluation device 158, such as a device for performing a color analysis of images captured by the imaging system 138 and/or any other image evaluation device 158, in order to determine one or more growth parameters from the images. Additionally or alternatively, one or more other types of growth parameters may be measured by the system 110. Preferably, the at least one growth parameter may be stored in the database 156 and/or any other database of the system 110. The database 156 may be stored in one or more storage devices 160 comprised in the system 110, such as in a system controller 126.

As disclosed above, the system 110 according to the present invention may be adapted to perform a method according to one or more of the different aspects of the present invention, preferably by using at least one contactless capacitive humidity sensor 132, preferably the at least one probe 134. Thus, the system 110 may be adapted to control the growth conditions, such as by simply monitoring the growth conditions of each plant container 112 or even by regulating the growth conditions for each of the plant containers 112.

Thus, the system 110, preferably the evaluation device 150, may be adapted to monitor the water consumption for each plant specimen 116. As outlined above, the water consumption may be used as an indicator for the physical condition of each plant specimen 116.

Further, additionally or alternatively, the system 110 may be used for tracking growth conditions for the plant specimens 116 comprised in the system 110. Therein, the system 110 may be used for controlling the humidity in each plant container 112 and for storing the humidity in a database, such as the database 156.

The system 110 may further be adapted to perform a tracking method, in which at least one drought test and/or at least one water use efficiency test is performed. Thus, by adjusting the amount of liquid supplied by the watering station 142 and/or the type of liquid supplied by the watering station 142, one or more tests may be performed, subjecting the plant specimens 116 to specific growth conditions. Thus, by reducing the amount of liquid, a drought test may be performed, and the response of the plant specimens 116 to this drought test may be monitored, such as by correlating the humidity measured by the measurement position 130 with the one or more growth parameters, such as one or more growth parameters measured by using the at least one measurement device 136. Further, the water consumption itself may be used as a growth parameter. Additionally or alternatively to the drought test, other types of tests may be performed, such as tests providing a reduced and/or increased amount of at least one type of salt or nutrient to the plant containers 112.

Further, the system and, preferably, the evaluation device 150 may be adapted or may be used for performing a method for breeding plants. In this method, the system 110 may be used to ensure that the liquid supply to the plant containers 112 is controlled. Further, the system 110 may be adapted to change the positions of the plant containers within the environment of the system 110 such that all plants substantially are uniformly exposed to the conditions in the environment. As outlined above, this might be performed by designing the transport system 118 as a closed loop transport system, such as by stepwise or continuously transporting the plant containers 112 into every possible position.

The system 110 may further be adapted to support and/or perform a method, in which plant specimens 116 are selected for further breeding or for commercial use. Thus, the system 110 may be adapted to compare phenotypic characteristics of the plant specimens. This comparison may be performed automatically, semi-automatically or manually, such as by evaluating at least one growth parameter of the plant specimens 116, such as the growth parameters stored in the database 156 of the system 110. Again, the system 110 may be adapted to successively transport the plant containers 112 to and from the measurement position 130 and for using the at least one contactless capacitive humidity sensor 132 for monitoring the humidity of the growing medium 114 in the plant containers 112.

The system 110 may further be adapted for performing a method for improved growing of plants for phenotyping, for selecting the most desired genotypes based on phenotype scoring. This method, again, may be performed automatically, semi-automatically or manually, such as by using the evaluation device 150. Thus, as outlined above, a displacement of the plants or plant specimens 116 may be performed by using the transport system 118.

Further, the method for improved growing may, again, comprise the measuring of the humidity of the growing medium 114 by using the contactless capacitive humidity sensor 132 and, preferably, a controlling of the humidity. With regard to potential embodiments of the measuring and controlling, reference may be made to the above-mentioned embodiments.

The system 110 according to FIGS. 1 and 2 may further be adapted for rapid analysis of stress resistance of growing plants. Thus, as outlined above, stress such as drought stress or salt stress or any other kind of stress or a combination of stresses may be applied to the plant specimens 116, automatically, semi-automatically or manually, by using the system 110, such as by appropriately controlling the water station 142 and/or the type of liquid supplied by the watering station 142.

The system 110 may be adapted to grow the plants or plant specimens 116 under stress conditions and for measuring the humidity of the growing medium 114 in the plant containers 112. The system 110 may further be adapted for analyzing the stress resistance on the plants, based on the humidity. Thus, as outlined above, the humidity may be an indicator of the water consumption of the plant specimens 116 and, thereby, be an indicator for the physiological condition of the plant specimens 116. Additionally or alternatively, the humidity itself may be part of the stress conditions. Again, as with the other methods according to the present invention, the method may fully or partially be implemented by using one or more software implementations, preferably software implementations in the data processing device 128.

With regard to the measurement principles that might be used by the probe 134, reference may be made to the description given above. Specifically, reference may be made to the publications on the capacitive humidity measurements.

Every material has a dielectric constant or relative permittivity, which may be measured by the probe 134. Water typically has a relative permittivity of approximately 80, whereas most other materials have a relative permittivity of approximately 1 to 10. Thus, the relative permittivity of sand, as an example, typically lies in the range between 3 and 4. Therefore, a large measurable difference exists between the relative permittivity of water and that of other types of materials, such as typical materials as used as a growing medium 114. The relative permittivity may be measured in absolute values and/or complex values.

The relative permittivity may be measured and correlated to a moisture value, thereby allowing for a determination of the humidity of the growing medium 114. The humidity may then be output by the probe 134, such as by an analogue and/or digital signal provided to the system controller 126. Thus, one or more measurement signals of the humidity measurement may be provided, such as standard signals of 0 to 10 VDC and/or 0 to 20 mA, from which measurement signals a direct humidity value may be derived and/or which may directly be used as a humidity value, such as a moisture content in mass percent. Typically, the more water or moisture is contained in the material of the growing medium 114, the closer the value of its relative permittivity is to 80.

In this or other embodiments of the present invention, the humidity measurement preferably may be performed as an online measurement, preferably as a real-time measurement. Thus, the growing medium 114 might pass the probe 134 in the measurement position 130. Alternatively or additionally, in this or in other embodiments of the present invention, another type of relative motion between the probe 134 and the plant container 112 might be used, such as a moving probe 134. In a real-time measurement, the measurement signal of the humidity measurement may be available instantly, even when fast-flowing products are measured. The measurement of solid bodies is also possible.

Preferably, an analogue output measurement signal is generated. Thus, an analogue output measurement signal of the humidity or the moisture measurement probe 134 of 0/2 . . . 10 VCD or 0/4 . . . 20 mA can be processed directly, preferably in a process sequence, and may be connected to a control, PC or PLC system, such as an appropriate system comprised in the system controller 126 or any other device of the system 110.

Depending on the type of material and its properties, the measuring probe 134 may reach different measurement depths. Preferably, the probe 134 is adapted to create an electric field in a dome-shaped region above the probe 134. Typically, the measurement depth reaches around 100 mm to 150 mm into the material of the growing medium 114. The total product moisture, i.e. the core moisture as well as the surface moisture of the material, i.e. the plant container 112 and the growing medium 114, may be analyzed. On account of this high penetration depth, soiling and minor deposits on the measurement surface may be insignificant.

The system 110 may be used in various applications, such as crop design or any other application, e.g. testing transgenic plants for the effect of a specific gene which is over- or underexpressed or even knocked down. Also, as outlined above, the system 110 may be used for pot moisture measurements in a drought experiment. All plant specimens 116 in a drought measurement may be transported constantly or successively into one or more measurement positions 130 comprising one or more probes 134. As such, transgenic plants may be tested on their drought resistance.

The system 110 may be adapted to monitor each individual plant specimen 116, preferably in such a way that water status and stress status of every individual plant specimen 116 may be monitored and preferably recorded. A rewatering can thus be accomplished for every single plant specimen 116 separately, such as by means of the at least one watering station 142, preferably in the vicinity or in connection to the at least one measurement position 130. Thus, an improvement over current drought tests may be achieved, since the latter typically only uses a batch, such as several hundred plant specimens 116 in one experiment. Typically, in these conventional drought tests, rewatering takes place at a moment in which the median pot water content reaches a certain value or, when the median stress level reaches a certain value. Thus, by using the system 110 and/or one or more of the methods disclosed above, the accuracy of the new drought test may be increased significantly.

Further, the system 110 may be adapted to calculate the water content dynamics of every single plant specimen 116 and/or plant container 112 separately. This may be used to provide a better insight into the physiological mechanisms acting on the individual plant specimens 116. The resolution of the screening for more water efficient plant specimens 116 may thus be taken to a higher level.

### Examples of Methods and Uses

In the following, exemplary embodiments of the methods and uses according to the present invention are disclosed. Specifically, the following provides an example for the astonishing finding according to the present invention that a population of plant specimens 116 having a low plant-to-plant variability may be provided by using drought conditions, preferably mild drought conditions, preferably at an early stage, preferably a pre-flowering stage. As an example of plant specimens 116, rice seedlings were used.

### 1. Introduction

Experiments for evaluating the impact of drought treatments were performed, specifically for evaluating the impact of an early drought onto the growth parameters of the plants. The early drought treatment consisted in two successive cycles of drought applied between seedling and early tillering stage. The primary purpose of this treatment was to screen for plants that tolerate drought at an early stage, as opposed e.g. to reproductive drought screens. Thus the purpose of these experiments was to find plants being tolerant such that these plants would either show a less important reduction of growth during drought, or a better capacity to recover, i.e. to resume growth after drought.

The protocol described below is designed to cause approx. 50% reduction in plant size, measured immediately after drought, as compared with well watered plants.

### 2. Protocol

Plants were sown, germinated, and selected for transplantation in the usual way, known to the skilled person. Standard pots and soil were used as plant containers and growing medium, respectively.

Plants were transplanted ten days after germination from sowing trays to the pots. Prior to transplantation, the soil in the pots was saturated to maximal capacity by prolonged sub-irrigation in order to reduce differences between pots.

The plants were not watered after transplantation. Instead, the water content was monitored through daily moisture measurements using a capacitance soil-moisture probe (Theta-Probe, Delta-T, UK). Alternatively or additionally, a contactless capacitive humidity sensor 132 might have been used.

For measuring an average humidity of the growing media in the plant containers, humidity measurements were made randomly in approx. 10% of the population of plants, and the average was calculated.

A first drought cycle was applied, comprising a drying of the soil. A re-watering was performed such that, when the average soil moisture reached 12% (water weight per unit of substrate weight), the plants were re-watered until the average moisture reached the maximum capacity (typically 60%).

The plants were then imaged to record the post-drought leaf area, as a potential example of a growth parameter.

A second cycle of drought was imposed in the same way, and the plants were imaged again after re-saturation of the soil.

From that point on, the plants were bred following a usual cultivation and evaluation protocol, generally known to the skilled person. However, any other breeding protocol might be used.

### 3. Results

In a preliminary experiment involving a small number of plants, the effect of early drought on basic plant growth parameters was determined. The results of this experiment are listed in Table 1.

TABLE 1
Effect of early drought on basic plant growth parameters as compared
to well-watered (normal, standard) conditions.

| Parameter | Early drought | Normal conditions | Penalty |
|---|---|---|---|
| Leaf area after drought | 8336 | 17513 | −52% |
| Leaf area after 1 week | 21150 | 30550 | −31% |
| recovery | | | |
| Leaf area after 2 weeks | 28959 | 36326 | −20% |
| recovery | | | |
| Final leaf area | 31981 | 36805 | −13% |
| (AreaMax) | | | |
| Fertility (fillrate) | 47 | 28 | 67% |
| Flowers per panicle | 40 | 48 | −18% |
| Harvest index | 80 | 55 | 45% |
| Nr filled seeds | 121 | 96 | 27% |
| Nr total seeds | 258 | 340 | −24% |
| Time to flowering (days) | 57 | 52 | 9% |
| Seed weight (TKW) | 21.5 | 21.2 | 1% |
| Seed yield (total | 2.6 | 2.0 | 28% |
| weight seeds) | | | |

The “normal” or standard conditions, as used in Table 1 as a comparison, were determined as follows:

Plants were grown in individual pots and each pot was provided daily with enough nutrient solution in order to reach the maximum retention capacity.

The growth parameters as listed in Table 1 have the following meanings and were determined as follows:
Leaf area after drought: Projected leaf area, as measured by horizontal digital imaging, immediately after the end of the drought treatment. Unit: mm²
Leaf area after 1 week recovery: Same as above, measured 1 week after return to normal watering conditions. Unit: mm²
Leaf area after 2 weeks recovery: Same as above, measured 2 weeks after return to normal watering conditions. Unit: mm²
Final leaf area (AreaMax): Measurement by weekly digital imaging of the maximum projected leaf area, inferred from a logistic curve fitting across weekly measurements throughout vegetative cycle. Unit: mm²
Fertility (fillrate): Ratio of number of filled seeds (=fertile seeds) over the total number of florets (filled+non filled) per plant. Measurement at harvest by automated seed counter. Unit: percentage.
Flowers per panicle: Total number of florets (filled+non filled) divided by the number of panicles. Measured at harvest by manual counting of panicles and automated seed counter.
Harvest index: Ratio of the total seed weight over “Final leaf area” (see above). Unit: grams/mm².
Nr filled seeds: Number of fertile seeds produced per plant, as opposed to “empty”, sterile seeds. Measurement at harvest by automated seed counter.
Nr total seeds: Total number of seeds (filled+non filled). Measurement at harvest by automated seed counter.
Time to flowering (days): Number of days between sowing and the emergence of the first panicle. Measurement by detection of panicle presence on weekly images. Unit: days.
Seed weight (TKW): Average weight per seed. Measured by automated seed counter and weighing. Unit: grams. 1000 seeds⁻¹
Seed yield (total weight seeds): Total weight of filled (fertile) seeds in grams.plant⁻¹

The “Penalty” in Table 1 was calculated as: (early drought−normal conditions)/normal conditions.

The primary effect of early drought was found to be a slowdown of plant growth as shown by “leaf area after drought” in Table 1. Upon return to normal conditions (recovery), the stressed plants catch up so that the final leaf area is only mildly affected by the treatment.

Other growth parameters were found to be reduced, such as the total number of seeds and the number of flowers per panicle.

On the other hand, many other growth parameters were found to be improved, such as fertility (ratio of filled versus non filled seeds), the final seed yield per plants and the harvest index. Flowering time was found to be delayed by 9% (5 days).

In further experiments, plant-to-plant variability was determined in plants subjected to early drought, and the plant-to-plant variability was compared with historical data of plants grown under normal conditions.

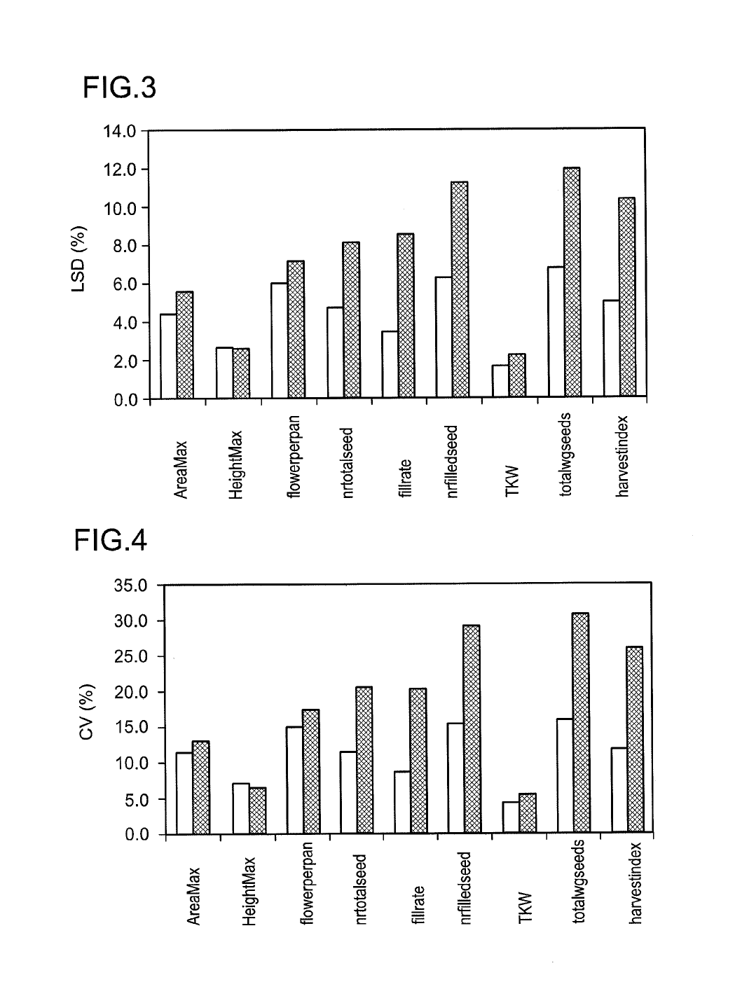

As measures for the plant-to-plant variability, coefficients of variation and least significant differences were used for various growth parameters of the plant specimens. The coefficient of variation (CV, standard deviation divided by the mean) and the least significant difference (LSD, smallest difference that remains statistically significant) were calculated for both conditions, i.e. for plants bred under drought conditions and under standard conditions. The results of these measurements are listed in Table 2 and in FIGS. 3 and 4.

TABLE 2
Comparison of variability of plant populations bred under early drought
conditions and of plant populations bred under standard conditions.

| | Coefficient | | | |
|---|---|---|---|---|
| | of variation (%) | | | |
| | Early | Normal | Least significant difference (%) | |
| | drought | conditions | Early drought | Normal conditions |
| Final leaf | 11.6 | 12.9 | 4.4 | 5.6 |
| area | | | | |
| Flowers per | 15.0 | 17.3 | 6.0 | 7.1 |
| panicle | | | | |
| Nr total | 11.4 | 20.4 | 4.7 | 8.1 |
| seeds | | | | |
| Fertility | 8.6 | 20.1 | 3.5 | 8.5 |
| Nr filled | 15.3 | 29.0 | 6.2 | 11.2 |
| seeds | | | | |
| Seed Weight | 4.1 | 5.3 | 1.7 | 2.2 |
| Total weight | 15.8 | 30.5 | 6.7 | 11.9 |
| seeds | | | | |
| Harvest | 11.6 | 25.7 | 5.0 | 10.3 |
| index | | | | |

In Table 2, the same parameter definitions as for Table 1 apply.

In FIGS. 3 and 4, open bars denote measurement values of plants bred under early drought conditions, whereas filled bars denote the corresponding measurement values of plants bred under normal (i.e. standard) conditions.

As a result, Table 2 and FIGS. 3 and 4 show a clear reduction of variability, mostly in the seed related parameters.

It is of particular interest that the LSD is reduced by 2-fold, which means that the resolution of the assay has been doubled. Therefore, using drought stress of similar intensity, or possibly milder, has the potential to greatly improve plant evaluation procedures by either providing finer resolution (smaller differences can be detected with same population size), or by allowing to reduce population size while maintaining the same level of accuracy.

### 3. Interpretation

The fact that an early drought stress reduces variability in parameters measured weeks later (such as fertility rate, seed yield, etc.) is surprising. Without intending to be bound by the following theories, different possible explanations of this effect may be as follows:
a) Plants evaporate water extracted from the soil. The speed at which the soil is depleted from its water is dependent on the transpiration capacity of the plant which itself is correlated to the leaf area. When water availability falls under a certain threshold, plants stop absorbing water from the soil and growth is inhibited. The bigger, fast growing, plants have higher transpiration capacity than small, slow growing plants. Therefore, in this setup, one may expect that bigger plants stop growing earlier, they undergo more heavy drought damage, and recover slower than smaller plants. Therefore, the initial difference in size and performance is reduced at later stages.
b) The relatively mild drought stress does not cause permanent injury to the plants, only a temporary arrest of growth. One can hypothesize that such mild stress induces acclimation to other types of stresses and/or induces a compensation response, therefore improving the overall performance of the plants. This hypothesis is supported by the observation that, upon return to normal conditions, the stressed plants exhibit faster growth, allowing them to catch up on the well-watered controls. Such acclimation and/or compensation responses have been observed in other plant systems and reported in literature.
c) The dry soil conditions during drought may result in better oxygenation of the root system and/or increase root production as an acclimation response. In both cases, the result is a healthier, more efficient root system which improves plant performance.

Summarizing, it was found that a relatively mild drought treatment, applied early in the growth cycle, reduces plant-to-plant variability at maturity and therefore allows to detect changes in yield components with greater accuracy or with reduced populations.

The drought treatment itself can possibly be applied in a different way than the way disclosed above. Instead of two successive drought cycles, as disclosed above, it is possible to use a different number of drought cycles, such as only one drought cycle. Further, instead of using cycles, other types of drought conditions may be applied. Further, the moisture threshold for re-watering might be higher (less severe) than 12%. Further, even though the experiments disclosed above were performed with rice, one may reasonably expect that this drought effect could be observed in other species, such as other cereals or other types of plants.

### REFERENCE NUMBERS

110 system for monitoring growth conditions
112 plant container
114 growing medium
116 plant specimen, plant
118 transport system
120 transport direction
122 transport belt
124 transport controller
126 system controller
128 data processing device
130 measurement position
132 contactless capacitive humidity sensor
134 probe
136 measurement device
138 imaging system
140 camera system
142 watering station
143 monitoring system
144 supply system
146 identifier
148 reader
150 evaluation device
152 display device
154 keyboard
156 database
158 image evaluation device
160 storage device

1-29. (canceled)

30. A system (110) for monitoring growth conditions of a plurality of plant containers (112), the system (110) having a transport system (118) for transporting the plant containers (112), each plant container (112) comprising at least one growing medium (114) and preferably at least one plant specimen (116), the system (110) further comprising at least one measurement position (130) having at least one contactless capacitive humidity sensor (132), the system (110) being adapted to successively transport the plant containers (112) to and from the measurement position (130), the system (110) further being adapted to measure the humidity of the growing medium (114) of the plant containers (112) in the measurement position (130) by using the contactless capacitive humidity sensor (132).

31. The system (110) of claim 30, wherein the contactless capacitive humidity sensor (132) is performing the humidity measurement from a lower side of the plant containers (112) through a bottom section of the plant containers (112).

32. The system (110) of claim 30, wherein the transport system (118) comprises a transport belt (122), and wherein the contactless capacitive humidity sensor (132) is mounted underneath the transport belt (122).

33. The system (110) of claim 30, the system (110) further having at least one watering station (142), the system (110) being adapted to add liquid to the growing medium (114) in each plant container (112).

34. The system (110) of claim 30, wherein the plant containers (112) each comprise at least one identifier (146), the system (110) being adapted to identify the plant container (112) presently being located in the measurement position (130).

35. The system (110) of claim 34, wherein the at least one identifier (146) comprises at least one barcode and/or at least one contactless electronic identifier (146).

36. The system (110) of claim 34, wherein the at least one identifier is at least one RFID tag.

37. The system (110) of claim 30, wherein the system (110) further comprises at least one monitoring system (143), the monitoring system (143) being adapted to monitor the humidity of the growing medium (114) in the plant containers (112).

38. The system (110) of claim 37, wherein the monitoring system (143) is adapted to monitor the humidity of the growing medium (114) in the plant containers (112) as a function of plant specimen (116) and/or as a function of time.

39. The system (110) of claim 30, wherein the system (110) further comprises at least one imaging system (138) for capturing images of the plant specimens (116).

40. The system (110) of claim 30, wherein the system (110) further comprises at least one measurement device (136) for measuring at least one growth parameter of the plant specimens (116).

41. The system (110) of claim 40, wherein the at least one growth parameter is selected from the group consisting of: a height of the plant specimen (116); a width of the plant specimen (116); a color parameter of the plant specimen (116); a number of leaves; at least one structure of the plant specimen (116); a presence of flowers in the plant specimen (116); a parameter characterizing the volume of the biomass of the plant specimen (116); a parameter characterizing the biochemical content of the plant specimen (116) and/or the growing medium (114) inside the plant container (112); and a parameter characterizing the root growth of the plant specimen (116).

42. A method for monitoring growth conditions of a plurality of plant containers (112), wherein each plant container (112) comprises at least one growing medium (114), wherein the plant containers (112) are successively transported to and from at least one measurement position (130), wherein the humidity of the growing medium (114) of the containers (112) in the measurement position (130) is measured by using at least one contactless capacitive humidity sensor (132).

43. The method of claim 42, wherein each plant container (112) further comprises at least one plant specimen (116).

44. A method for monitoring growth conditions of a plurality of plant containers (112), wherein each plant container (112) comprises at least one growing medium (114), wherein the plant containers (112) are successively transported to and from at least one measurement position (130), wherein the humidity of the growing medium (114) of the containers (112) in the measurement position (130) is measured by using at least one contactless capacitive humidity sensor (132), and wherein the system (110) of claim 30 is used for monitoring growth conditions of the plurality of plant containers (112).

45. The method of claim 42, wherein a water consumption of each plant specimen (116) is monitored.

46. A tracking method for tracking growth conditions of a plurality of plant specimens (116), wherein the plurality of plant specimens (116) are growing in growing medium (114) inside a plurality of plant containers (112), wherein the method of claim 42 is used for monitoring the humidity in each plant container (112), wherein the humidity in each plant container (112) is stored in a database (156).

47. The tracking method of claim 46, wherein the humidity in each plant container (112) is stored in a database (156) as a function of time and/or as a function of plant specimen (116).

48. The tracking method of claim 46, wherein at least one growth parameter for each plant specimen (116) is recorded in the database (156).

49. The tracking method of claim 48, wherein the at least one growth parameter for each plant specimen (116) is recorded in the database (156) as a function of time and/or as a function of plant specimen (116).

50. The tracking method of claim 46, wherein a drought test and/or a water use efficiency test is performed in which a variety of plant specimens (116) are subjected to a lack or reduced amount of water over a period of time, wherein the plant specimens' (116) reaction to the lack of water or reduced amount of water is recorded.

51. A method for breeding plants (116) comprising growing a plurality of plants (116) of at least one species in a plurality of plant containers (112) charged with growing medium (114) of uniform characteristics in an environment of controlled climatic conditions, with controlled supply of liquid and changing the positions of the plant containers (112) within the environment as required to ensure at least substantially uniform exposure of all plants (116) in the plant containers (112) to conditions in the environment, and which process further comprises the step of selecting plants (116) for further breeding or for commercial use by comparing the phenotypic characteristics of the plants (116), wherein the plant containers (112) are successively transported to and from a measurement position (130) by a transport system (118), wherein the humidity of the growing medium (114) of the plant containers (112) in the measurement position (130) is measured by using at least one contactless capacitive humidity sensor (132).

52. A method for improved growing of plants (116) for phenotyping, for selecting the most desired genotypes based on phenotype scoring, the method comprising:
displacing the plants (116) automatically during their growing cycle so as to avoid extended exposure to a particular micro-environment;
measuring a humidity of a growing medium (114) of the plants (116) by using at least one contactless capacitive humidity sensor (132); and
controlling the humidity.

53. A method for rapid analysis of stress resistance of growing plants (116), comprising:
growing the plants (116) under stress conditions;
measuring a humidity of a growing medium (114) of the plants (116) by using at least one contactless capacitive humidity sensor (132); and
analyzing the stress resistance of the plants (116) based on the humidity.

54. A method for providing a population of plant specimens (116) comprising:
determining standard watering conditions leading to a predetermined breeding result;
determining drought conditions including watering conditions below the standard watering conditions; and
breeding a population of plant specimens (116) in at least one plant container comprising at least one growing medium, by using the drought conditions.

55. The method of claim 54, wherein, during breeding of the population of plant specimens (116), a contactless capacitive humidity sensor (132) is used for monitoring the drought conditions.

56. The method of claim 54, wherein the breeding of plant specimens (116) takes place by using the drought conditions before flowering of the plant specimens.

57. The method of claim 56, wherein after flowering of the plant specimens (116), standard watering conditions are used.

58. The method of claim 54, wherein the drought conditions comprise a watering of the growing medium such that the growing medium is watered up to at least one predetermined upper level, wherein a re-watering is performed as soon as a humidity of the growing medium has decreased to at least one predetermined lower level, wherein the drought conditions comprise at least two drought cycles, wherein in each cycle a watering up to the at least one predetermined upper level and a subsequent decrease down to the at least one predetermined lower level takes place.

59. The method of claim 54, wherein the drought conditions comprise a watering of the growing medium to a time-averaged value of 20% to 80% as compared to the standard conditions.

60. The method of claim 59, wherein the drought conditions comprise a watering of the growing medium to a time-averaged value of 40% to 70% as compared to the standard conditions.

61. A population of plant specimens (116) produced by the method of claim 54.

62. A method for determining the phenotypic effect of at least one effector condition, comprising subjecting the population of plant specimens (116) of claim 61 to the at least one effector condition and determining at least one growth parameter of the plant specimens (116).

63. The method of claim 62, wherein at least two plant specimens (116) of the population are subjected to different effector conditions, wherein the growth parameters of the at least two plant specimens (116) are compared.
